(19) United States
(12) Patent Application Publication
(10) Pub. No.: US 2009/0215714 A1
(43) Pub. Date: Aug. 27, 2009

(54) Pyrimidines, such as cytidine, in treatments for patients with biopolar disorder

(76) Inventors: Perry Renshaw, Salt Lake City, UT (US); Bruce Cohen, Lexington, MA (US)

Correspondence Address: CLARK & ELBING LLP, 101 FEDERAL STREET, BOSTON, MA, 02110, US

(21) Appl. No.: 11/629,110

(22) PCT Filed: Jun. 10, 2005
(86) PCT No.: PCT/US05/20682

§ 371 Date: Oct. 17, 2008

Related U.S. Application Data
(60) Provisional application No. 60/579,175, filed on Jun. 10, 2004.

(51) Int. Cl. A61P 25/24; A61K 31/7068

(52) U.S. Cl. 514/47; 514/46

(57) ABSTRACT

The invention is based on the discovery that specific dosages of pyrimidine compositions, such as cytidine compositions, can be used to treat patients diagnosed with bipolar disorder.

### CROSS REFERENCE TO RELATED APPLICATION

This application claims priority, under 35 U.S.C. §119(e), from U.S. Provisional Patent Application Ser. No. 60/579,175 filed on Jun. 10, 2004, which is incorporated herein by reference in its entirety.

### TECHNICAL FIELD

This invention relates to the treatment of bipolar disorder.

### BACKGROUND

Bipolar disorder, which is also referred to as manic-depression, is a brain disorder that causes extreme shifts in a person's mood, thought, energy, behavior, and ability to function. The symptoms of bipolar disorder can be are severe, and can result in emotional problems, poor job or school performance, and even suicide. The name “bipolar” comes from the patients' mood swings, which can alternate between the “poles” of mania (highs) and depression (lows). These mood swings can be quite dramatic, from overly “high” and/or irritable to sad and hopeless, and then back again, often with periods of normal mood in between, and severe changes in energy and behavior go along with these changes in mood. Bipolar disorder typically develops in late adolescence or early adulthood. However, some people have their first symptoms during childhood or late in life. This disorder is not always viewed as an illness, and people may suffer for years before proper diagnosis.

Bipolar disorder has been separated into two categories, Type I and Type II, and is typically diagnosed following the guidelines in the Diagnostic and Statistical Manual of Mental Disorders (DSM) Fourth Edition, 1994 (American Psychiatric Association, 1400 K Street NW, Suite 1101, Washington, D.C. 20005-2403 USA). The fourth edition of these guidelines, DSM-IV, identifies the diagnostic features of Bipolar I Disorder as follows.

Bipolar I Disorder (DSM-IV, p. 350)

This disorder is a clinical course that is characterized by the occurrence of one or more Manic Episodes or Mixed Episodes. Often individuals have also had one or more Major Depressive Episodes. Episodes of Substance-Induced Mood Disorder (due to the direct effects of a medication, or other somatic treatments for depression, a drug of abuse, or toxin exposure) or of Mood Disorder Due to a General Medical Condition do not count toward a diagnosis of Bipolar I Disorder. In addition, the episodes are not better accounted for by Schizoaffective Disorder and are not superimposed on Schizophrenia, Schizophreniform Disorder, Delusional Disorder, or Psychotic Disorder Not Otherwise Specified.

The diagnostic features of Bipolar II Disorder are as follows.

Bipolar II Disorder (DSM-IV, p. 359)

This disorder is a clinical course that is characterized by the occurrence of one or more Major Depressive Episodes accompanied by at least one Hypomanic Episode. Hypomanic Episodes should not be confused with the several days of euthymia that may follow remission of a Major Depressive Episode. Episodes of Substance-Induced Mood Disorder (due to the direct effects of a medication, or other somatic treatments for depression, a drug of abuse, or toxin exposure) or of Mood Disorder Due to a General Medical Condition do not count toward a diagnosis of Bipolar I Disorder. In addition, the episodes are not better accounted for by Schizoaffective Disorder and are not superimposed on Schizophrenia, Schizophreniform Disorder, Delusional Disorder, or Psychotic Disorder Not Otherwise Specified.

The following diagnostic criteria, also from the DSM-IV apply.

Criteria for Major Depressive Episode (DSM-IV, p. 327)

A. Five (or more) of the following symptoms have been present during the same 2-week period and represent a change from previous functioning; at least one of the symptoms is either (1) depressed mood or (2) loss of interest or pleasure. Symptoms that are clearly due to a general medical condition, or mood-incongruent delusions or hallucinations should not be included.
Depressed mood most of the day, nearly every day, as indicated by either subjective report (e.g., feels sad or empty) or observation made by others (e.g., appears tearful). In children and adolescents, the mood can be an irritable mood.
Markedly diminished interest or pleasure in all, or almost all, activities most of the day, nearly every day (as indicated by either subjective account or observation made by others)
Significant weight loss when not dieting or weight gain (e.g., a change of more than 5% of body weight in a month), or decrease or increase in appetite nearly every day. In children, consider failure to make expected weight gains.
Insomnia or hypersomnia nearly every day.
Psychomotor agitation or retardation nearly every day (observable by others, not merely subjective feelings of restlessness or being slowed down).
Fatigue or loss of energy nearly every day.
Feelings of worthlessness or excessive or inappropriate guilt (which may be delusional) nearly every day (not merely self-reproach or guilt about being sick).
Diminished ability to think or concentrate, or indecisiveness, nearly every day (either by subjective account or as observed by others).
Recurrent thoughts of death (not just fear of dying), recurrent suicidal ideation without a specific plan, or a suicide attempt or a specific plan for committing suicide.

B. The symptoms do not meet criteria for a Mixed Episode.

C. The symptoms cause clinically significant distress or impairment in social, occupational, or other important areas of functioning.

D. The symptoms are not due to the direct physiological effects of a substance (e.g., a drug of abuse, a medication) or a general medical condition (e.g., hypothyroidism).

E. The symptoms are not better accounted for by bereavement, i.e., after the loss of a loved one, the symptoms persist for longer than 2 months or are characterized by marked functional impairment, morbid preoccupation with worthlessness, suicidal ideation, psychotic symptoms, or psychomotor retardation.

Criteria for Manic Episode (DSM-IV, p. 332)

A. A distinct period of abnormally and persistently elevated, expansive, or irritable mood, lasting at least 1 week (or any duration if hospitalization is necessary).

B. During the period of mood disturbance, three (or more) of the following symptoms have persisted (four if the mood is only irritable) and have been present to a significant degree:
Inflated self-esteem or grandiosity.
Decreased need for sleep (e.g., feels rested after only 3 hours of sleep).
More talkative than usual or pressure to keep talking.
Flight of ideas or subjective experience that thoughts are racing. Distractibility (i.e., attention too easily drawn to unimportant or irrelevant external stimuli).
Increase in goal-directed activity (either socially, at work or school, or sexually) or psychomotor agitation.
Excessive involvement in pleasurable activities that have a high potential for painful consequences (e.g., engaging in unrestrained buying sprees, sexual indiscretions, or foolish business investments).

C. The symptoms do not meet criteria for a Mixed Episode.

D. The mood disturbance is sufficiently severe to cause marked impairment in occupational functioning or in usual social activities or relationships with others, or to necessitate hospitalization to prevent harm to self or others, or there are psychotic features.

E. The symptoms are not due to the direct physiological effects of a substance (e.g., a drug of abuse, a medication, or other treatments) or a general medical condition (e.g., hyperthyroidism).

Manic-like episodes that are clearly caused by somatic antidepressant treatment (e.g., medication, electroconvulsive therapy, light therapy) should not count toward a diagnosis of Bipolar I Disorder.

Criteria for Mixed Episode (DSM-IV, p. 335)

A. The criteria are met both for a Manic Episode and for a Major Depressive Episode (except for duration) nearly every day during at least a 1-week period.

B. The mood disturbance is sufficiently severe to cause marked impairment in occupational functioning or in usual social activities or relationships with others, or to necessitate hospitalization to prevent harm to self or others, or there are psychotic features.

C. The symptoms are not due to the direct physiological effects of a substance (e.g., a drug of abuse, a medication, or other treatment) or a general medical condition (e.g., hyperthyroidism).

Criteria for Hypomanic Episode (DSM-IV, p. 338)

A. A distinct period of persistently elevated, expansive, or irritable mood, lasting throughout at least 4 days, that is clearly different from the usual nondepressed mood.

B. During the period of mood disturbance, three (or more) of the following symptoms have persisted (four if the mood is only irritable) and have been present to a significant degree:
Inflated self-esteem or grandiosity.
Decreased need for sleep (e.g., feels rested after only 3 hours of sleep).
More talkative than usual or pressure to keep talking.
Flight of ideas or subjective experience that thoughts are racing.
Distractibility (i.e., attention too easily drawn to unimportant or irrelevant external stimuli).
Increase in goal-directed activity (either socially, at work or school, or sexually) or psychomotor agitation.
Excessive involvement in pleasurable activities that have a high potential for painful consequences (e.g., engaging in unrestrained buying sprees, sexual indiscretions, or foolish business investments).

C. The episode is associated with an unequivocal change in functioning that is uncharacteristic of the person when not symptomatic.

D. The disturbance in mood and the change in functioning are observable by others.

E. The episode is not severe enough to cause marked impairment in social or occupational functioning, or to necessitate hospitalization, and there are no psychotic features.

F. The symptoms are not due to the direct physiological effects of a substance (e.g., a drug of abuse, a medication, or other treatment) or a general medical condition (e.g., hyperthyroidism).

Hypomanic-like episodes that are clearly caused by somatic antidepressant treatment (e.g., medication, electroconvulsive therapy, light therapy) should not count toward a diagnosis of Bipolar II Disorder.

Current drug therapy for bipolar disorder includes the use of lithium or valproic acid; however side effects are frequent and troublesome, and patients do not respond fully, leading to frequent recurrences of mania and depression.

### SUMMARY

The invention is based, in part, on the discovery that individuals who are diagnosed with one or more symptoms of bipolar disorder can be treated with specific dosages of pyrimidines, such as cytidine. In particular, the invention is based on a human clinical trial of cytidine in patients with bipolar disorder.

Thus, in general, the invention features a method of treating an individual diagnosed as having one or more symptoms of bipolar disorder by administering to the individual an effective amount of a pyrimidine composition, such as a cytidine composition. The individual may have one or more symptoms of bipolar disorder. For example, the amount of the cytidine composition can be effective to improve one or more of the symptoms of bipolar disorder. For example, the effective amount of the cytidine composition can provide about 100 to 5000 mg/day, e.g., about 250, 500, 750, 1000, 2000, 3000, or 4000 milligrams/day, or 1 to 50 mg of cytidine/kg of body weight/day, e.g., 3 to 10 mg/kg of body weight/day. The cytidine composition can be administered orally, for example, when the cytidine composition includes cytidine or acylated derivatives of cytidine, such as triacetyl cytidine, and a liquid ingestible carrier.

A pyrimidine composition is either a purified pyrimidine, a compound or product that contains a pyrimidine, a compound that increases the level of a pyrimidine in the patient, or a compound or molecule that mimics the biological function of a pyrimidine. Such a compound can be a pyrimidine precursor or prodrug, which is processed, e.g., metabolized, degraded, or cleaved, in the body to form a pyrimidine. Such a compound can also be a pyrimidine derivative, which includes pyrimidine, and other molecules or compounds bound (e.g., covalently or non-covalently) to a pyrimidine, but that do not impair the pyrimidine's biological activity in patients with increased purine levels. Such compounds can also be pyrimidine mimetics, such as other nucleotides or small molecules that have a sufficiently similar three-dimensional shape or electron configuration that the compound has at least 50 percent of the biological activity of the pyrimidine. Such compounds can also be drugs or other compounds that induce the body to produce one or more pyrimidines.

For example, a pyrimidine composition can be a cytidine composition. A cytidine composition is either a purified cytidine, a compound or product that contains cytidine, a compound that increases the level of cytidine in the patient, or a compound or molecule that mimics the biological function of cytidine. Such a compound can be a cytidine precursor or prodrug, which is processed, e.g., metabolized, degraded, or cleaved, in the body to form cytidine. Such a compound can also be a cytidine derivative, which includes cytidine, and other molecules or compounds bound (e.g., covalently or non-covalently) to cytidine, but that do not impair cytidine's biological activity in patients with increased purine levels. Such compounds can also be cytidine mimetics, such as other nucleotides or small molecules that have a sufficiently similar three-dimensional shape or electron configuration that the compound has at least 50 percent of the biological; activity of cytidine. Such compounds can also be drugs or other compounds that induce the body to produce cytidine.

In one aspect, the invention includes a method of treating an individual exhibiting one or more symptoms of bipolar disorder (e.g., type I or type II bipolar disorder) by administering to the individual an effective amount of a cytidine composition. Cytidine compositions can include purified cytidine, CDP-choline, citicoline, cytidine monophosphate (CMP), cytidine diphosphate(CDP), cytidine triphosphate(CTP), deoxy-CMP, deoxy-CDP, or deoxy-CTP.

In another embodiment, the method of treating an individual exhibiting one or more symptoms of bipolar disorder includes administering the cytidine composition in combination with an antimanic drug. In some embodiments, an effective amount of the cytidine composition is an amount sufficient to improve one or more symptoms of bipolar disorder, e.g., one or more symptoms of depression, one or more symptoms of a manic episode, one or more symptoms of a mixed episode, or one or more symptoms of a hypomanic episode.

In another aspect, the invention includes a method of reducing the severity of a major depressive episode in an individual who is suffering from bipolar disorder, and the method comprising administering to the individual an effective amount of a cytidine composition. In some embodiments, the method includes administering the cytidine composition in combination with another treatment for bipolar disorder, e.g., an antimanic drug.

By “treating” is meant the medical management of a patient to cure, ameliorate, or prevent a specific disorder. This term includes active treatment directed towards improvement of a disorder, and causal treatment directed towards the removal of a cause of the disorder. In addition, this term includes palliative treatment designed for the relief of one or more symptoms rather than curing the disorder; preventive treatment directed to prevention of the disorder; and supportive treatment employed to supplement another specific therapy directed toward the improvement of the disorder.

By “therapeutically-effective amount” is meant an amount of a cytidine composition sufficient to produce a healing, curative, prophylactic, stabilizing, or ameliorative effect in the treatment of bipolar disorder. Such an effect is sufficient even if it improves only one symptom in a patient.

The new methods provide a safe therapy for bipolar disorder, without the side effect of mania, which can accompany other known treatments.

Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF FIGURE DESCRIPTION

FIG. 1 is a graph comparing the depression of bipolar subjects treated with DEPAKOTE® and cytidine with the depression of bipolar subjects treated with DEPAKOTE® and placebo.

### DETAILED DESCRIPTION

The new methods are based on the finding that individuals diagnosed with bipolar disorder can benefit from treatment by the administration of a pyrimidine, such as cytidine, prodrugs of cytidine, and cytidine analogs. The patient can be easily treated by the administration of an effective amount of a pyrimidine composition such as a cytidine composition, for example, by oral or systemic intravenous administration.

The new methods are based on a double-blind, long-term clinical trial designed to assess the impact of cytidine on patients with bipolar disorder who were undergoing a major depressive episode. The results demonstrate that combined administration of oral cytidine with divalproex sodium (DEPAKOTE®) in this patient population improved the symptoms of depression to a greater extent than combined administration of placebo with DEPAKOTE®. The trial began with 39 patients, of which 19 were treated with cytidine, and 20 were treated with placebo. As the trial continued, a total of 65 subjects were enrolled in the trial, of which 33 were treated with cytidine, and 32 were treated with placebo.

### General Methods of Therapy

The new methods involve the administration of an effective amount of a pyrimidine composition, such as a cytidine composition, to a patient diagnosed with one or more symptoms of bipolar disorder. The cytidine composition can be formulated into a therapeutic composition and administered using a variety of known routes of administration, and in various dosage forms.

To formulate pharmaceutical grade therapeutic compositions, the cytidine composition can be purified by standard methods, e.g., filtration, to remove contaminants, if present. The final compositions can be lyophilized and resuspended in sterile, deionized water before further compounding. The therapeutic compositions can be formulated as solutions, suspensions, suppositories, tablets, granules, powders, capsules, ointments, or creams. In the preparation of these compositions, at least one pharmaceutical excipient can be included. Examples of pharmaceutical excipients include solvents (e.g., water or physiological saline), solubilizing agents (e.g., polysorbates, or Cremophor EL7), agents for achieving isotonicity, preservatives, antioxidizing agents, lactose, starch, crystalline cellulose, mannitol, maltose, calcium hydrogen phosphate, light silicic acid anhydride, calcium carbonate, binders (e.g., starch, polyvinylpyrrolidone, hydroxypropyl cellulose, ethyl cellulose, carboxy methyl cellulose, or gum arabic), lubricants (e.g., magnesium stearate, talc, or hardened oils), or stabilizers (e.g., lactose, mannitol, maltose, polysorbates, macrogols, or polyoxyethylene hardened castor oils). If desired, glycerin, dimethylacetamide, 70% sodium lactate, surfactant, or basic substances such as sodium hydroxide, ethylenediamine, ethanolamine, sodium bicarbonate, arginine, meglumine, or trisaminomethane can be added. Common disintegrants that can be included in the composition include croscarmellose sodium, crospovidone, gellan gum, and sodium starch glycolate.

When the pyrimidine composition, e.g., a cytidine composition, is ingested, the excipient or carrier can be water, a flavored beverage such as a fruit juice, broth, carbonated beverage, milk, or milk shake.

Biodegradable polymers such as poly-D,Lactide-co-glycolide or polyglycolide can be used as a bulk matrix if slow release of the composition is desired (see, e.g., U.S. Pat. Nos. 5,417,986, 4,675,381, and 4,450,150). Pharmaceutical preparations such as solutions, tablets, granules or capsules can be formed with these components. If the composition is to be administered orally, flavorings and/or colors can be added.

The new compositions can be administered via any appropriate route, e.g., intravenously, intraarterially, topically, transdermally, by injection, intraperitoneally, intrapleurally, orally, subcutaneously, intramuscularly, sublingually, nasally, by inhalation, intraepidermally, or rectally, using standard techniques.

Dosages administered in practicing the new methods will depend on factors including the specific cytidine composition used and its concentration in the composition, the mode and frequency of administration, the age, weight, sex, and general health of the subject, and the severity of the autistic symptoms. In general, the new compositions can be administered in amounts ranging between 1 mg and 70 mg of cytidine per kilogram of body weight per day, e.g., 2, 3, 5, 10, 15, 20, 25, 30, 35, 40, 45, 50, 55, 60, or more mg/kg/day. The general dosage is between 3 and 40 mg/kg/day, which amounts to 200 to 4000 mg (e.g., 500, 750, 1000, 1500, 2000, 2500, 3000, 3500, or 3750 mg) per patient per day. Oral tablets of cytidine can be used. The daily dosage is administered on an ongoing basis until symptoms subside.

Dosages can be administered with meals or once, twice, or more times per day to achieve the best relief of symptoms. The dosage should be adjusted to provide a reduction in symptoms. Once the proper dosage is, it can be easily maintained over time as required.

Administration is repeated as necessary, as determined by one skilled in the art. By varying the amount of the composition or dosage, the administration protocol can be optimized based on the present disclosure to elicit a maximal improvement in symptoms of bipolar disorder. Physicians, pharmacologists, and other skilled artisans are able to determine the most therapeutically effective treatment regimen, which will vary from patient to patient. The potency of a specific composition and its duration of action can require administration on an infrequent basis, including administration in an implant made from a polymer that allows slow release of the cytidine.

Skilled artisans are also aware that the treatment regimen must be commensurate with issues of safety and possible toxic effect produced by the cytidine or other components in the compositions. Thus, before administering the above compositions to humans, toxicity testing can be conducted in animals. In an example of toxicity testing, the cytidine compositions can be administered to mice via an oral or parenteral route with varying dosages of cytidine in the composition, and the mice observed for signs of toxicity using standard techniques. Previous studies suggest that cytidine is very well tolerated.

### Cytidine Compositions

A cytidine composition is either purified cytidine, a compound or product that contains cytidine, a compound that increases the level of cytidine in the patient, or a compound or molecule that mimics the biological function of cytidine. Such a compound can be a cytidine precursor or prodrug, which is processed, e.g., metabolized, degraded, or cleaved, in the body to form cytidine. Such a compound can also be a cytidine derivative, which includes cytidine, and other molecules or compounds bound (e.g., covalently or non-covalently) to cytidine, but that do not impair cytidine's biological activity in patients with increased purine levels. Such compounds can also be cytidine mimetics, such as other nucleotides or small molecules that have a sufficiently similar three-dimensional shape or electron configuration that the compound has at least 50 percent of the biological activity of cytidine. Such compounds can also be drugs or other compounds that induce the body to produce cytidine, or drugs or compounds that inhibit degradation or otherwise prolong the half-life of cytidine in the body.

Cytidine precursors or prodrugs include cytidine monophosphate (CMP), cytidine diphosphate (CDP), cytidine triphosphate (CTP), as well as mono-, di-, or tri-esters of cytidine including triacetyl cytidine. Other precursors and prodrugs include CDP-choline and cytidine 5′-diphosphocholine, frequently prepared as cytidine 5′-diphosphocholine and also known as citicoline. Cytidine mimetics include uridine, mono-, di- or tri-esters of uridine, including mono-, di-, and triacetyl uridine, and mono, di- or tri-phosphates of uridine including uridine monophosphate. mono-, di-, or tri-esters of cytidine including triacetyl cytidine. Deoxy-versions of these and other ribonucleosides may also be useful, e.g., dCMP, dCDP, or dCTP.

Cytidine compositions also include encapsulated cytidine, e.g., liposome- or polymer-encapsulated cytidine. Cytidine compositions also include cytidine linked (e.g., covalently or non-covalently) to various antibodies, ligands, or other targeting and enveloping or shielding agents (e.g., albumin or dextrose), to allow the cytidine to reach the target site (e.g., the central nervous system, muscle cells, or the peripheral nervous system) prior to being removed from the blood stream, e.g., by the kidneys and liver, and prior to being degraded.

Cytidine salts or food products containing cytidine that transform into cytidine upon administration to a host such as human can also be used.

Useful cytidine-containing compounds include, without limitation, any compound comprising cytidine, CMP, CDP; and CTP. Cytidine and cytidine-containing compounds and analogs are well tolerated in humans.

Combination with Other Therapeutics

The pyrimidine, e.g., cytidine, compositions described herein can be administered as a monotherapy, as combinations of two or more different pyrimidines, e.g., cytidine compositions (or uridine and cytidine compositions), or in combination with other compounds for the treatment of bipolar disorders.

For example, the pyrimidine compositions can be administered in conjunction with lower doses of current treatments for bipolar disorder, including stimulants and antidepressants. For example, divalproex sodium (DEPAKOTE®) has been used to treat bipolar disorder.

In particular examples, the pyrimidine compositions may be administered in combination with an antidepressant, anticonvulsant, antianxiety, antimanic, antipyschotic, antiobsessional, sedative, stimulant, or anti-hypertensive medication. Examples of these medications include, serotonin reuptake inhibitors, monoamine oxidase inhibitors, tricyclic antidepressants, dopamine agonists (e.g., bromocriptine, pergolide), bupropion, venlafaxine, nefazodone, benzodiazepine, trazodone, lithium, risperidone, topiramate, lamotrigine, gabapentin, nimodipine, divalproex, quetiapine, divalproex, lamotrigine, carbamazepine, clozapine, olanzapine, topiramate, thyroid hormone (e.g., T3 or T4), Omega-3 fatty acids, calcium channel blockers (other than nimodipine), tiagabine, cholinesterase inhibitors, tamoxifen, and phenytoin.

### EXAMPLES

The invention is further described in the following examples, which do not limit the scope of the invention described in the claims.

### Example 1

### Patient Study

65 subjects with either bipolar disorder I or II, as determined by the Structured Clinical Interview for DSM-IV-Patient Version (SCID), were studied during a depressive episode, as determined by a Hamilton Depression Rating Scale (HAM-D) score of 16 or greater. Following baseline assessment, subjects were randomized into treatment with DEPAKOTE® (divalproex sodium) plus oral cytidine supplementation (33 subjects) or Depakote plus placebo (32 subjects). Purified cytidine was administered to subjects in gel capsules, twice daily, at a dose of 1 gram/day. HAM-D scores for every subject were evaluated and recorded once a week for twelve weeks following baseline assessment.

During the twelve weeks of study, no subjects exhibited treatment emergent mania according to recently defined criteria of a Young Mania Rating Scale (YMRS) score of less than 15 at baseline and a score of 15 or greater anytime during the study. Tohen et al., (2003) Arch. Gen. Psychiatry, 60:1079-88. Using modified criteria (i.e., YMRS score of less than 12 at baseline and a score of greater than 12 subsequently), no subjects in the cytidine group and two subjects in the placebo group met the criteria for treatment emergent mania. Using a cutoff of YMRS score of 10, one subject receiving cytidine and three subjects receiving placebo met the criteria for treatment emergent hypomania.

The HAM-D scale is a 17-item scale used in the art to assess the severity of depression in patients already diagnosed with an affective disorder. Higher scores indicate more severe depression. Questions posed to subjects relate to symptoms such as depressed mood, guilty feelings, suicide, sleep disturbances, anxiety levels, and weight loss. Hamilton, (1960) J. Neurology and Neurosurgery in Psychiatry, 23:56-62.

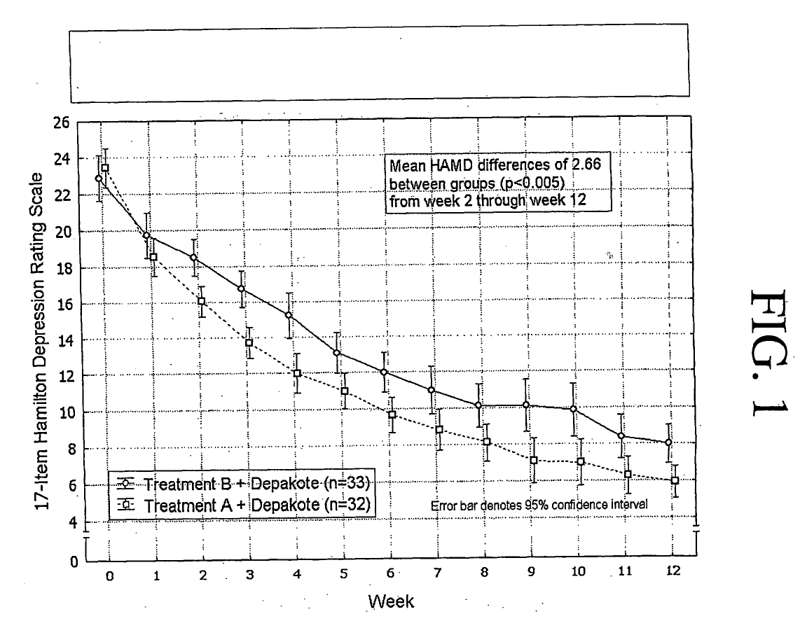

FIG. 1 shows that during the study the cytidine supplementation enhanced the affective improvement of patients during the study compared to placebo supplementation.

Our findings suggest enhanced affective improvement from week 2 through week 12 for cytidine compared to placebo as an add-on to divalproex treatment for subjects with bipolar depression, as shown by a significant difference in HAM-D scores. The mean difference in HAM-D scores between groups was 2.66±0.65 from week 2 through 12 (FIG. 1). The cytidine group exhibited a 17.8 point (75%) decrease in mean HAM-D-17 score from baseline to week 12, and the placebo group exhibited a 14.8 point (65%) decrease. Although these data suggest a high placebo response, it is more likely that this lessening of depressive symptoms was due to the divalproex treatment that was administered to both groups.

The entire subject group evidenced a 57% decrease (22.2 to 9.6) in mean 28-item HAM-D (HAM-D-28) score from baseline to week 12, and the medication-naïve group alone (n=11) exhibited a 68% decrease (20.6 to 6.6).

This cytidine trial exhibited a 10% difference in antidepressant efficacy between treatment groups; consequently, it may be similarly interpreted that the addition of oral cytidine to divalproex in the treatment of bipolar depression appears to result in a 10% increase in antidepressant efficacy compared to the administration of divalproex alone.

Less Frequent Treatment—Emergent Manic Symptoms:

No subject out of the 33 in the cytidine arm of the study and one subject out of the 32 in the placebo arm exhibited treatment-emergent mania using recently defined criteria of a YMRS score of less than 15 at baseline and greater than a score of 15 anytime during the study. Using modified criteria (YMRS of less than 12 at baseline and a score greater than 12 subsequently), one subject in the cytidine group and four subjects in the placebo group met the criteria. For a cutoff of YMRS score of 9, one subject receiving cytidine and six subjects receiving placebo met the criteria for treatment-emergent hypomania during a 12-week trial period (Chi-square test, df=1, X²=4.18, p=0.041). The current result indicates that the subjects taking divalproex and cytidine have reduced low-level treatment-emergent hypomanic symptoms compared to those taking divalproex and placebo.

### Example 2

### Magnetic Resonance Spectroscopy (MRS) Studies

A companion imaging single voxel ¹H MRS study to the behavioral study described in Example 1 examined Glx and Lac changes in frontal gray matter. Nineteen subjects from the cytidine group, 17 subjects from the placebo group, and 14 healthy comparison subjects were scanned at baseline, week 2, and week 4. Brain MRS was performed using a 3.0 Tesla GE whole body imaging system (GE VH/i, USA). The localized MR spectra were recorded using a point-resolved spatially localized spectroscopy (PRESS) sequence on a General Electric 3T scanner, which was optimized for acquisition of ¹H MR spectra from the frontal lobe. Voxel size was 1.5×1.5×1.5 cm. The voxels were verified in scout views in T2 images obtained vertically in the Z-axis to include predominantly gray matter of the anterior cingulate for the frontal brain region. The acquisition parameters were: repetition time (TR), 2000 ms; echo time (TE), 35 ms; average=128, phase size-cycling=8, and acquisition time, 4 minutes 16 seconds. Results from these analyses are shown in Table 1 (concentrations units are millimolar).

Subjects with bipolar depression had increased Glu and Gln levels compared to healthy comparison subjects at baseline (12.9%, p=0.095 and 23.2%, p=0.038, respectively). Subjects with bipolar depression had decreased GABA levels (−20.3%, p=0.051) at baseline. Bipolar depressed subjects also had increased Lac levels (10.6%, p=0.063) (see Table 1).

Both the cytidine and placebo groups showed 22.2% and 3.6% decreases in Glu levels over the 4-week period (p<0.01 and p=0.327, respectively). For Gln levels, the cytidine and placebo groups showed 5.3% and 6.9% decreases over the 4-week period (p=0.241 and p=0.083, respectively). For GABA levels, the cytidine and placebo groups showed 13.2% and 7.9% increases over the 4 week period (p=0.032 and p=0.073, respectively). For Lac levels, the cytidine and placebo groups showed 17.0% and 1.8% decreases over the 4 week period (p=0.032 and p=0.562, respectively; see Table 1).

Untreated bipolar depressed subjects had increased Glu, Gln, and Lac levels and decreased GABA levels relative to healthy comparison subjects. While levels of these brain metabolites changed toward control values with both cytidine and placebo, those changes were strongly related to cytidine, i.e., there were significant decreases in Glu and Lac levels in the cytidine arm, as opposed to the placebo arm.

TABLE 1

| Cytidine (n = 19) | | Placebo (n = 17) | | Control (n = 14) | |
|---|---|---|---|---|---|
| Mean | SD | Mean | SD | Mean | SD |

Changes in Gray Matter Glu Concentrations

| | | | | | | |
|---|---|---|---|---|---|---|
| Baseline | 11.38 | 2.04 | 11.24 | 2.72 | 10.02 | 1.89 |
| Week 2 | 9.18 | 2.12 | 11.31 | 2.34 | 10.35 | 1.93 |
| Week 4 | 8.85 | 1.68 | 10.83 | 1.86 | 9.87 | 1.76 |

Changes in gray matter Gln concentrations

| | | | | | | |
|---|---|---|---|---|---|---|
| Baseline | 7.38 | 1.86 | 7.43 | 2.35 | 6.01 | 2.01 |
| Week 2 | 7.21 | 2.31 | 6.83 | 1.94 | 6.15 | 1.91 |
| Week 4 | 6.99 | 1.57 | 6.92 | 2.03 | 6.26 | 2.14 |

Changes in gray matter GABA concentrations

| | | | | | | |
|---|---|---|---|---|---|---|
| Baseline | 0.91 | 0.41 | 0.89 | 0.33 | 1.13 | 0.35 |
| Week 2 | 0.97 | 0.38 | 0.89 | 0.24 | 1.08 | 0.29 |
| Week 4 | 1.03 | 0.26 | 0.96 | 0.29 | 1.05 | 0.33 |

Changes in gray matter Lac concentrations

| | | | | | | |
|---|---|---|---|---|---|---|
| Baseline | 1.71 | 1.16 | 1.65 | 0.87 | 1.52 | 1.02 |
| Week 2 | 1.44 | 1.08 | 1.56 | 1.05 | 1.61 | 0.83 |
| Week 4 | 1.42 | 0.91 | 1.62 | 0.65 | 1.46 | 0.75 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating an individual exhibiting one or more symptoms of bipolar disorder, the method comprising administering to the individual an effective amount of a cytidine composition.

2. The method of claim 1, wherein an effective amount of the cytidine composition provides about 1 to 50 mg of a cytidine composition/kg of body weight/day.

3. The method of claim 1, wherein an effective amount of the cytidine composition provides about 3 to 10 mg of a cytidine composition/kg of body weight/day.

4. The method of claim 1, wherein the individual is diagnosed under DSM-IV guidelines as having biopolar disorder and wherein an effective amount of the cytidine composition comprises between 250 and 1000 mg of cytidine/day.

5. The method of claim 4, wherein the cytidine composition is administered in combination with an antimanic drug.

6. The method of claim 1, wherein the cytidine composition comprises purified cytidine.

7. The method of claim 1, wherein the cytidine composition comprises CDP-choline or citicoline.

8. The method of claim 1, wherein the composition comprises cytidine monophosphate (CMP), cytidine diphosphate (CDP), cytidine triphosphate (CTP), deoxy-CMP, deoxy-CDP, or deoxy-CTP.

9. A method of claim 1, wherein an effective amount of the cytidine composition is administered orally.

10. The method of claim 1, wherein the individual is suffering from type I bipolar disorder.

11. The method of claim 1, wherein the individual is suffering from type II bipolar disorder.

12. The method of claim 1, wherein an effective amount of the cytidine composition is an amount sufficient to improve one or more symptoms of bipolar disorder.

13. The method of claim 12, wherein the symptoms are one or more symptoms of a major depressive episode.

14. The method of claim 12, wherein the symptoms are one or more symptoms of a manic episode.

15. The method of claim 12, wherein the symptoms are one or more symptoms of a mixed episode.

16. The method of claim 12, wherein the symptoms are one or more symptoms of a hypomanic episode.

17. A method of reducing the severity of depression in an individual suffering from bipolar disorder, the method comprising administering to the individual an effective amount of a cytidine composition.

18. The method of claim 17, further comprising administering the cytidine composition in combination with another treatment of bipolar disorder.

19. The method of claim 17, further comprising administering the cytidine composition in combination with an antimanic drug.

20. A method of reducing anxiety in an individual suffering from bipolar disorder, the method comprising administering to the individual an effective amount of a cytidine composition.

21. A method of reducing the severity of manic symptoms in an individual in need of treatment for bipolar disorder, the method comprising administering to the individual an effective amount of the cytidine composition.

22.-27. (canceled)
